(19) United States
(12) Patent Application Publication
(10) Pub. No.: US 2025/0086785 A1
(43) Pub. Date: Mar. 13, 2025

(54) HIGH-QUALITY EMBEDDINGS FOR MEDICAL IMAGING AND SMALL, EASY-TO-TRAIN NETWORKS FOR LOW-DATA TASKS

(71) Applicant: GOOGLE LLC, Mountain View, CA (US)

(72) Inventors: Andrew Beckmann SELLERGREN, El Cerrito, CA (US); Dilip KRISHNAN, Needham, MA (US); Shravya Ramesh SHETTY, San Francisco, CA (US)

(21) Appl. No.: 18/292,498

(22) PCT Filed: Jul. 18, 2022
(86) PCT No.: PCT/US2022/037494

§ 371 Date: Jan. 26, 2024

Related U.S. Application Data
(60) Provisional application No. 63/228,981, filed on Aug. 3, 2021.

(51) Int. Cl. G06T 7/00; G06N 20/00

(52) U.S. Cl. CPC G06T 7/0012; G06N 20/00; G06T 2207/20081; G06T 2207/20084

(57) ABSTRACT

Generation of high-performance machine learning models often requires significant computational resources and access to extensive training datasets. This makes development of such models for rare or novel diseases, where diagnostic imagery or other training data is limited, difficult. Methods are provided to apply extensive generic medical imagery training datasets to train machine learning models to embed input medical imaging data into generically informative embedding spaces. Relatively smaller training datasets specific to a novel or rare disease can then be used to develop high-performance models by updating the parameters of the pre-trained generic model and/or by training a smaller, task-specific model to predict one or more variables of interest based on embedding vectors output from the pre-trained generic model. The functionality of such a generic model can be made available via an online service to facilitate development of such task-specific models by smaller research groups.

### CROSS-REFERENCE TO RELATED APPLICATIONS

The present application is a non-provisional patent application claiming priority to U.S. Provisional Patent Application No. 63/228,981, filed Aug. 3, 2021, the contents of which are hereby incorporated by reference.

### BACKGROUND

It is desirable in many tasks to use available training data (e.g., medical images and related information) to train a machine learning model (e.g., an artificial neural network) to classify inputs or to generate some other prediction or output based on inputs. In practice, larger models (e.g., having more trainable parameters) are able to achieve higher performance (e.g., sensitivity, specificity). However, the training of such larger models often requires more training examples and for the set of training examples to be ‘higher quality’ (e.g., spanning a wider variety of potential inputs and outputs in a manner that is less biased).

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1 illustrates aspects of an example system.

FIG. 2 illustrates a flowchart of an example method.

FIG. 3 illustrates a flowchart of an example method.

FIG. 4 illustrates a flowchart of an example method.

FIG. 5 illustrates a flowchart of an example method.

FIG. 6 illustrates a flowchart of an example method.

FIG. 7 illustrates a flowchart of an example machine learning model training and inference process.

FIG. 8A illustrates a flowchart of an example method.

FIG. 8B depicts experimental results.

FIG. 9 depicts summary information about a training dataset.

FIG. 10 depicts summary information about a training dataset.

FIG. 11 depicts summary information about a training dataset.

FIG. 12A depicts experimental results.

FIG. 12B depicts experimental results.

FIG. 13 depicts experimental results.

FIG. 14 depicts experimental results.

FIG. 15 depicts experimental results.

FIG. 16 depicts experimental results.

### DETAILED DESCRIPTION

### I. Overview

A number of very large machine learning models have been developed (e.g., EfficientNet, ResNet-BiT) that match or exceed human performance in a variety of image classification tasks (e.g., identifying abnormal contents of or generating a diagnosis or other treatment information from a chest X-ray or other medical diagnostic imaging information). These models can also provide such classification or other information more quickly and more cheaply than human radiologists or other human classifiers.

However, current models are very large (e.g., having many millions or billions of trainable parameters) and training these models often requires extensive training datasets of thousands or millions of images. Further, the computational task of training and/or executing such models can be very expensive and difficult to accomplish, requiring extensive amounts of memory, computational cycles, information storage and transfer bandwidth, and other resources. The process of training such models (e.g., manually selecting features, manually managing hyperparameter searches, creating and applying heuristic model elements) can also involve extensive effort by highly-trained computer scientists.

These factors increase the cost and difficulty of applying state-of-the-art image processing models to novel tasks, rendering those models out of reach to smaller research projects or teams. These limitations are exacerbated when the object is to apply the models to diseases, processes, or conditions that are rare or that are changing quickly, since the amount of available training data in such circumstances is likely to be limited (e.g., when attempting to predict optimal treatments or treatment outcomes for a pandemic disease like COVID-19 which mutates, for which the standard treatment is rapidly developing, and whose target population may be changing with the development and rollout of vaccines or other preventive measures).

Embodiments provided herein address these issues in two ways. In a first aspect, a ‘generic’ medical image model is generated (e.g., from an existing model trained on natural images) that can receive a medical diagnostic image (e.g., a chest X-ray) and output an output vector that represents the input image in a multi-dimensional embedding space (e.g., an embedding space that has several hundred or several thousand dimensions). Such a generic model can be trained on available low-cost generic training sets that contain many training images and associated medical records, labels (e.g., labels that indicate whether medical diagnostic images represent ‘normal’ or ‘abnormal’ anatomy), or other data that can be used to train the generic model to output embeddings that can be useful for classifying medical diagnostic images. In a second aspect, such a generic model can be trained and maintained on a central computing system (e.g., a cloud computing system) that can then service requests from other systems (e.g., a physician's laptop, a radiologist's workstation) for model-related data (e.g., output multi-dimensional embeddings) for novel diagnostic medical images.

Such model-related data could be output vectors representing the novel images in the multi-dimensional embedding space. Alternatively, the central system (and/or the remote system(s)) could also include further models (e.g., a further trained version of the generic model, or a smaller model that receives the output vectors of the generic model as an input) to which it could apply the novel images in order to output diagnoses, treatments, prognoses, condition or disorder severity values, or other predictions or information related to a specific condition or diagnosis. By training the generic machine learning model with a large but generically medically relevant training set, a “specific” machine learning model related to a specific condition or diagnosis of interest can be developed therefrom using a relatively small number of training images (e.g., only a few dozen or a few hundred) while still obtaining high levels of specificity and accuracy. Such a specific machine learning model could include an updated version of the generic machine learning model and/or a “cap” model that receives the output vector of the generic machine learning model and outputs a diagnosis, prognosis, process or condition severity value, or other information indicative of property or presence of the specific condition or diagnosis.

Such embodiments provide a number of benefits. One benefit relates to the logistics and cost of applying such large-scale models to novel medical datasets. Training, maintaining, and executing such large-scale models on a central computing system allows for the economic costs of utilizing such a model on a case-by-case basis to be reduced. This cost reduction can be related to the ability of many smaller groups (e.g., research groups, hospitals, universities) to derive benefit from using such a model a relatively smaller number of times, allowing the cost of training and maintaining the generic model to be spread. Additionally, the existence of such a central model resource allows such groups to avoid the significant infrastructural costs of developing a computational system with sufficient memory, computational resources, information storage and bandwidth, and other resources sufficient to train and/or execute such a model.

Another benefit of such embodiments is increased protection of the privacy of individuals whose medical diagnostic images are applied to the model. In some examples, only the bare medical diagnostic image is sent to the central system, and only the embedding output vector is returned, without any identifying information being transmitted between the systems. The embedding output can then be used locally by the requestor system (e.g., by executing a local classifier on the output) to generate a diagnosis or other information for the medical diagnostic image. Indeed, privacy can also be enhanced during the training of a specific machine learning model for a specific condition or diagnosis by using the central system to provide multi-dimensional embeddings for a set of training images. The multi-dimensional embeddings, along with classifier labels or other training data, can then be used locally by a local system (e.g., a computer of a university or hospital research group) to train a classifier that can then be used to predict a diagnosis or other information for medical diagnostic images based on multi-dimensional embeddings determined therefor by the central system. In this way, sensitive classifier labels, medical records, or other training information can be kept secure on the local system, with only the medical diagnostic images being transmitted to the central system. Such a central model service could also have benefits with respect to compressing medical diagnostic images, by projecting the information from the images into the multi-dimensional embedding space.

Yet another benefit of such embodiments is the ability to generate highly specific and accurate predictive machine learning models for novel and/or quickly changing conditions or diagnoses based on relatively small training data sets. This is due to leveraging the high-quality, generically medically relevant multi-dimensional embedding generated by the “generic” machine learning model, which has been trained using a relatively much larger set of training images and data related to a wide variety of medical conditions or diagnoses. Starting from such a pre-trained generic model allows high-quality specific models to be trained using only dozens or hundreds of training examples. This is especially beneficial for rare conditions/diagnoses, conditions/diagnoses for which training examples are particularly expensive to access (e.g., due to a higher cost of specialty care necessary to conclusively diagnose, quantify, rate, or otherwise classify the specific condition or diagnosis), and/or conditions or diagnoses whose treatment or presentation changes quickly over time, limiting the amount of relevant data available to train a predictive model.

Generating a specific trained machine learning model for a specific condition or diagnosis could include applying training examples (medical diagnostic images and corresponding labels or other data) to the generic trained machine learning model to update the parameters of the generic trained machine learning model. Additionally or alternatively, a linear classifier, a nonlinear classifier, a decision tree, a regression tree, an artificial neural network, a convolutional neural network, a support vector machine, or some other form of “cap” machine learning model could be trained to accept the output vector of the generic trained machine learning model and to output a diagnosis, condition severity value, or other indication of a property or presence of the specific condition of diagnosis. Such a “cap” machine learning model can be generated by the central computing system that maintains the generic trained machine learning model. Alternatively, a remote computing system could generate the cap machine learning model. This could be done in order to further protect the privacy of the training data, e.g., by keeping the medical records, diagnoses, classifier labels, or other data associated with the training data private on a local computing system and only transmitting the medical diagnostic images to the central computing system. The output vectors determined by the central system and received by the local system could then be used, in combination with the other training data, to train the cap machine learning model.

Such a specific trained machine learning model (e.g., the updated generic trained machine learning model and/or one or more ‘cap’ machine learning models the received output vectors therefrom) could then be periodically updated and/or re-generated using newly available data. This could be done in order to update the prediction of the models as a condition or diagnosis changes over time (e.g. due to mutation of a pathogen, improvements in the treatment(s) for a specific condition or diagnosis, changes in a population affected by the condition or diagnosis, changes in vaccine distribution or public health policies related to the condition or diagnosis). By reducing the amount of training data needed to generate a “specific” trained machine learning model from a pre-trained, high-quality “generic” trained machine learning model, the embodiments described herein permit such model updates to be performed at a higher rate, allowing the most recently-generated specific model update to more accurately reflect current conditions.

The generic trained machine learning model could be generated in a variety of ways. In some examples, the generic trained machine learning model could be developed from a precursor trained machine learning model by updating the precursor trained machine learning model using medical diagnostic images and other generic medical diagnostic training data. Such a precursor trained machine learning model could have been trained using a training set of natural images, e.g., an EfficientNet-B7 model, with 66 million parameters, trained using the hundreds of thousands of natural images of the ImageNet training dataset. In another example, a ResNet-101x3 model or a ResNet-152x4 model (with 401 million and 981 million parameters, respectively) could be trained using the tens of millions of natural images available in the JFT-300M training dataset.

As noted above, training sets that include medical diagnostic images (e.g., chest X-rays) and associated training label data can be expensive to acquire. This issue is exacerbated when training especially large models, as many training examples are necessary. Accordingly, the methods described herein include automatically generating training labels for available medical diagnostic images by applying natural language processing or other techniques to automatically generate labels for images based on the contents of medical records (including free text notes) associated with the images. In an example, a label could be determined for each medical diagnostic image in a training dataset that classifies the images as either ‘normal’ or ‘abnormal.’ Such a simplified labeling scheme simplifies label generation, increases the accuracy of the automatically generated labels, and allows for medical diagnostic images from a variety of different sources (e.g., relating to a variety of different treatment centers, presenting conditions, etc.) to be aggregated together without additional dataset processing. Such training datasets can also be augmented by rotating some of the medical diagnostic images, horizontally flipping some of the medical diagnostic images, blanking randomly-selected portions of some of the medical diagnostic images, or performing some other data augmentation processes on the training dataset.

A supervised contrastive loss function can be used to generate the pretrained generic machine learning model. Such a loss function can be useful to move together groups of similarly-classified images (e.g., ‘normal’ images) within the multi-dimensional embedding space while breaking apart groups of dissimilarly-classified images within the multi-dimensional embedding space. Such a loss function is also useful when using ‘noisy’ training labels, like those that might be generated using natural language processing or other methods to automatically extract labels from free text notes or other medical record data associated with medical diagnostic images. In contrast with other uses of the supervised contrastive loss function, higher temperature parameter values resulted in improved results when training based on automatically-generated ‘normal’/‘abnormal’ classifier labels. Temperature parameter values greater than 0.5 (e.g., between 0.7 and 0.8) were found to result in improved models (e.g., improved trained EfficientNet-B7 models).

### II. Illustrative Systems

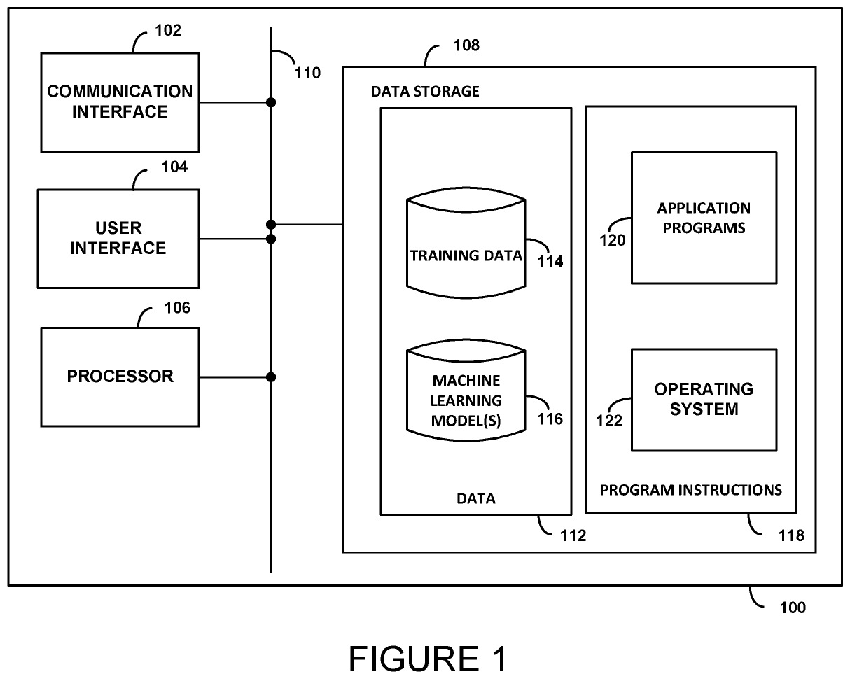

FIG. 1 illustrates an example computing system 100 that may be used to implement the methods described herein. By way of example and without limitation, computing system 100 may be a cellular mobile telephone (e.g., a smartphone), a computer (such as a desktop, notebook, tablet, or handheld computer, a server), elements of a cloud computing system, a robot, a drone, an autonomous vehicle, or some other type of device. It should be understood that computing system 100 may represent a physical computing device such as a server, a particular physical hardware platform on which a machine learning application operates in software, or other combinations of hardware and software that are configured to carry out machine learning functions as described herein. The computing system 100 could be a central system (e.g., a server, elements of a cloud computing system) that is configured to receive medical diagnostic images or other information (e.g., medical records, diagnostic information, class labels, or other information related to the images) from a remote system (e.g., a computing system in a physician's or radiologist's office or clinic) and to responsively transmit, to that remote system, output vector embeddings, diagnoses, or other information generated by applying the medical diagnostic image(s) to a machine learning model as described herein. Additionally or alternatively, the computing system 100 could be such a remote system, configured to transmit medical diagnostic images to a central system, receive output vectors, diagnostic information, or other information in response, and/or to take some other actions as described herein (e.g., to apply a received output vector to a linear, nonlinear, or other variety of classifier to generate a diagnosis or other prediction about a medical diagnostic image).

As shown in FIG. 1, computing system 100 may include a communication interface 102, a user interface 104, a processor 106, and data storage 108, all of which may be communicatively linked together by a system bus, network, or other connection mechanism 110.

Communication interface 102 may function to allow computing system 100 to communicate, using analog or digital modulation of electric, magnetic, electromagnetic, optical, or other signals, with other devices, access networks, and/or transport networks. Thus, communication interface 102 may facilitate circuit-switched and/or packet-switched communication, such as plain old telephone service (POTS) communication and/or Internet protocol (IP) or other packetized communication. For instance, communication interface 102 may include a chipset and antenna arranged for wireless communication with a radio access network or an access point. Also, communication interface 102 may take the form of or include a wireline interface, such as an Ethernet, Universal Serial Bus (USB), or High-Definition Multimedia Interface (HDMI) port. Communication interface 102 may also take the form of or include a wireless interface, such as a Wifi, BLUETOOTH®, global positioning system (GPS), or wide-area wireless interface (e.g., WiMAX or 3GPP Long-Term Evolution (LTE)). However, other forms of physical layer interfaces and other types of standard or proprietary communication protocols may be used over communication interface 102. Furthermore, communication interface 102 may comprise multiple physical communication interfaces (e.g., a Wifi interface, a BLUETOOTH® interface, and a wide-area wireless interface).

In some embodiments, communication interface 102 may function to allow computing system 100 to communicate with other devices, remote servers, access networks, and/or transport networks.

User interface 104 may function to allow computing system 100 to interact with a user or other entity, for example to receive input from and/or to provide output to the user. Thus, user interface 104 may include input components such as a keypad, keyboard, touch-sensitive or presence-sensitive panel, computer mouse, trackball, joystick, microphone, and so on. User interface 104 may also include one or more output components such as a display screen which, for example, may be combined with a presence-sensitive panel. The display screen may be based on CRT, LCD, and/or LED technologies, or other technologies now known or later developed. User interface 104 may also be configured to generate audible output(s), via a speaker, speaker jack, audio output port, audio output device, earphones, and/or other similar devices.

Processor 106 may comprise one or more general purpose processors—e.g., microprocessors—and/or one or more special purpose processors—e.g., digital signal processors (DSPs), graphics processing units (GPUs), floating point units (FPUs), network processors, tensor processing units (TPUs), or application-specific integrated circuits (ASICs). In some instances, special purpose processors may be capable of image processing, image alignment, merging images, transforming images, executing machine learning models, training machine learning models, among other applications or functions. Data storage 108 may include one or more volatile and/or non-volatile storage components, such as magnetic, optical, flash, or organic storage, and may be integrated in whole or in part with processor 106. Data storage 108 may include removable and/or non-removable components.

Processor 106 may be capable of executing program instructions 118 (e.g., compiled or non-compiled program logic and/or machine code) stored in data storage 108 to carry out the various functions described herein. Therefore, data storage 108 may include a non-transitory computer-readable medium, having stored thereon program instructions that, upon execution by computing system 100, cause computing system 100 to carry out any of the methods, processes, or functions disclosed in this specification and/or the accompanying drawings. The execution of program instructions 118 by processor 106 may result in processor 106 using data 112.

By way of example, program instructions 118 may include an operating system 122 (e.g., an operating system kernel, device driver(s), and/or other modules) and one or more application programs 120 (e.g., functions for executing and/or training a machine learning model) installed on computing system 100. Data 112 may include training data (e.g. medical diagnostic images and associated labels, medical records, etc.) 114 and/or machine learning model(s) 116 that may be determined therefrom or obtained in some other manner.

Application programs 120 may communicate with operating system 122 through one or more application programming interfaces (APIs). These APIs may facilitate, for instance, application programs 120 transmitting or receiving information via communication interface 102, receiving and/or displaying information on user interface 104, and so on.

Application programs 120 may take the form of “apps” that could be downloadable to computing system 100 through one or more online application stores or application markets (via, e.g., the communication interface 102). However, application programs can also be installed on computing system 100 in other ways, such as via a web browser or through a physical interface (e.g., a USB port) of the computing system 100.

### III. Example Methods

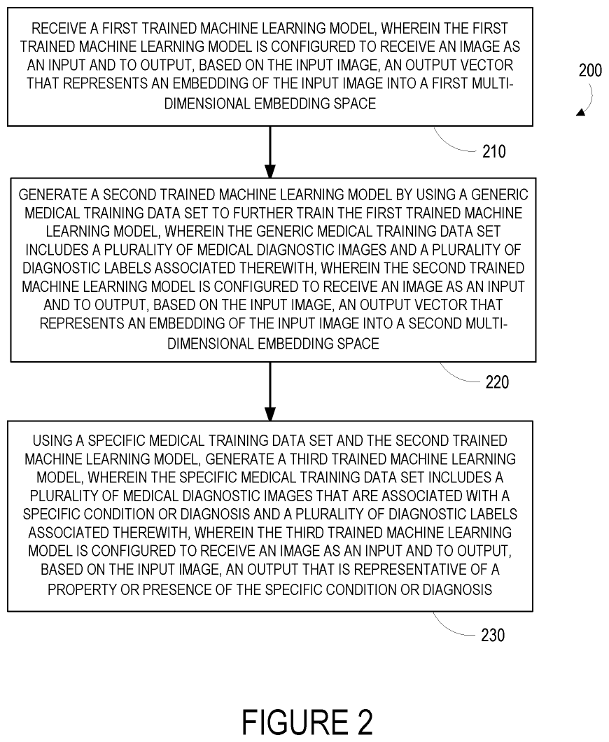

FIG. 2 is a flowchart of an example computer-implemented method 200. The method 200 includes receiving a first trained machine learning model, wherein the first trained machine learning model is configured to receive an image as an input and to output, based on the input image, an output vector that represents an embedding of the input image into a first multi-dimensional embedding space (210). The method 200 additionally includes generating a second trained machine learning model by using a generic medical training data set to further train the first trained machine learning model, wherein the generic medical training data set includes a plurality of medical diagnostic images and a plurality of diagnostic labels associated therewith, wherein the second trained machine learning model is configured to receive an image as an input and to output, based on the input image, an output vector that represents an embedding of the input image into a second multi-dimensional embedding space (220). The method 200 additionally includes, using a specific medical training data set and the second trained machine learning model, generating a third trained machine learning model, wherein the specific medical training data set includes a plurality of medical diagnostic images that are associated with a specific condition or diagnosis and a plurality of diagnostic labels associated therewith, wherein the third trained machine learning model is configured to receive an image as an input and to output, based on the input image, an output that is representative of a property or presence of the specific condition or diagnosis (230). The method 200 could include additional or alternative features.

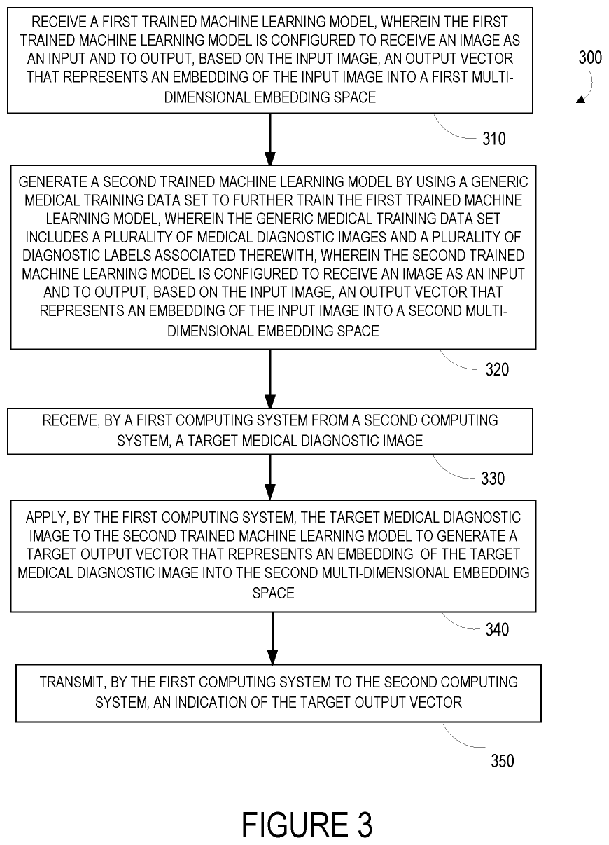

FIG. 3 is a flowchart of an example computer-implemented method 300. The method 300 includes receiving a first trained machine learning model, wherein the first trained machine learning model is configured to receive an image as an input and to output, based on the input image, an output vector that represents an embedding of the input image into a first multi-dimensional embedding space (310). The method 300 additionally includes generating a second trained machine learning model by using a generic medical training data set to further train the first trained machine learning model, wherein the generic medical training data set includes a plurality of medical diagnostic images and a plurality of diagnostic labels associated therewith, wherein the second trained machine learning model is configured to receive an image as an input and to output, based on the input image, an output vector that represents an embedding of the input image into a second multi-dimensional embedding space (320). The method 300 additionally includes receiving, by a first computing system from a second computing system, a target medical diagnostic image (330). The method 300 additionally includes applying, by the first computing system, the target medical diagnostic image to the second trained machine learning model to generate a target output vector that represents an embedding of the target medical diagnostic image into the second multi-dimensional embedding space (340). The method 300 also includes transmitting, by the first computing system to the second computing system, an indication of the target output vector (350). The method 300 could include additional or alternative features.

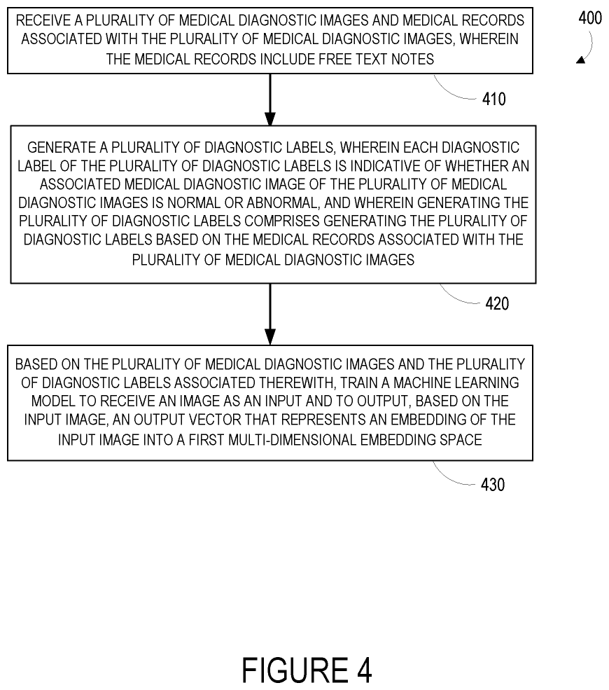

FIG. 4 is a flowchart of an example computer-implemented method 400. The method 400 includes receiving a plurality of medical diagnostic images and medical records associated with the plurality of medical diagnostic images, wherein the medical records include free text notes (410). The method 400 additionally includes generating a plurality of diagnostic labels, wherein each diagnostic label of the plurality of diagnostic labels is indicative of whether an associated medical diagnostic image of the plurality of medical diagnostic images is normal or abnormal, and wherein generating the plurality of diagnostic labels comprises generating the plurality of diagnostic labels based on the medical records associated with the plurality of medical diagnostic images (420). The method 400 additionally includes, based on the plurality of medical diagnostic images and the plurality of diagnostic labels associated therewith, training a machine learning model to receive an image as an input and to output, based on the input image, an output vector that represents an embedding of the input image into a first multi-dimensional embedding space (430). The method 400 could include additional or alternative features.

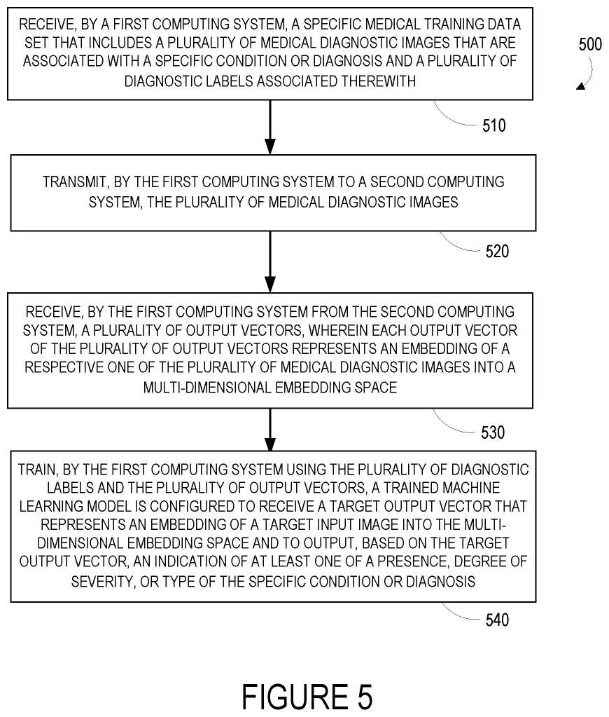

FIG. 5 is a flowchart of an example computer-implemented method 500. The method 500 includes receiving, by a first computing system, a specific medical training data set that includes a plurality of medical diagnostic images that are associated with a specific condition or diagnosis and a plurality of diagnostic labels associated therewith (510). The method 300 additionally includes transmitting, by the first computing system to a second computing system, the plurality of medical diagnostic images (520). The method 500 additionally includes receiving, by the first computing system from the second computing system, a plurality of output vectors, wherein each output vector of the plurality of output vectors represents an embedding of a respective one of the plurality of medical diagnostic images into a multi-dimensional embedding space (530). The method 500 additionally includes training, by the first computing system using the plurality of diagnostic labels and the plurality of output vectors, a trained machine learning model is configured to receive a target output vector that represents an embedding of a target input image into the multi-dimensional embedding space and to output, based on the target output vector, an indication of at least one of a presence, degree of severity, or type of the specific condition or diagnosis (540). The method 500 could include additional or alternative features.

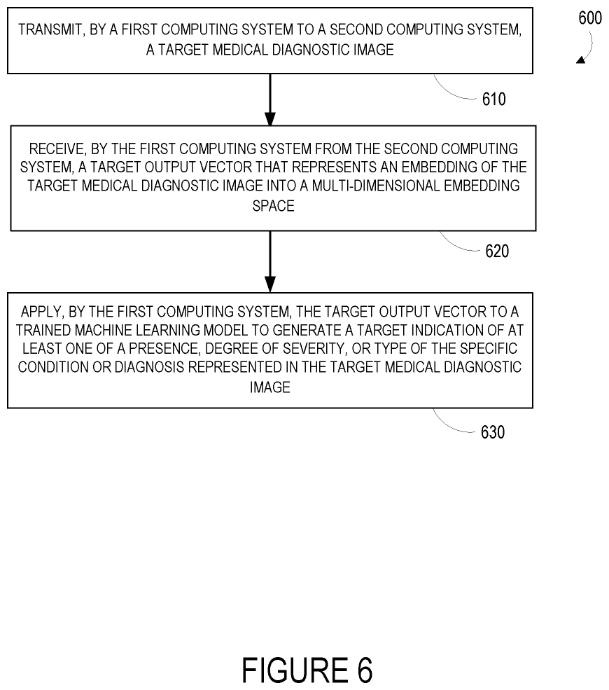

FIG. 6 is a flowchart of an example computer-implemented method 600. The method 600 includes transmitting, by a first computing system to a second computing system, a target medical diagnostic image (610). The method 600 additionally includes receiving, by the first computing system from the second computing system, a target output vector that represents an embedding of the target medical diagnostic image into a multi-dimensional embedding space (620). The method 600 additionally includes applying, by the first computing system, the target output vector to a trained machine learning model to generate a target indication of at least one of a presence, degree of severity, or type of the specific condition or diagnosis represented in the target medical diagnostic image (630). The method 600 could include additional or alternative features.

### IV. Example Machine Learning Models and Training Thereof

A machine learning model as described herein may include, but is not limited to: an artificial neural network (e.g., a herein-described convolutional neural networks, a recurrent neural network, a Bayesian network, a hidden Markov model, a Markov decision process, a logistic regression function, a support vector machine, a suitable statistical machine learning algorithm, and/or a heuristic machine learning system), a support vector machine, a regression tree, an ensemble of regression trees (also referred to as a regression forest), a decision tree, an ensemble of decision trees (also referred to as a decision forest), or some other machine learning model architecture or combination of architectures.

An artificial neural network (ANN) could be configured in a variety of ways. For example, the ANN could include two or more layers, could include units having linear, logarithmic, or otherwise-specified output functions, could include fully or otherwise-connected neurons, could include recurrent and/or feed-forward connections between neurons in different layers, could include filters or other elements to process input information and/or information passing between layers, or could be configured in some other way to facilitate the generation of predicted color palettes based on input images.

An ANN could include one or more filters that could be applied to the input and the outputs of such filters could then be applied to the inputs of one or more neurons of the ANN. For example, such an ANN could be or could include a convolutional neural network (CNN). Convolutional neural networks are a variety of ANNs that are configured to facilitate ANN-based classification or other processing based on images or other large-dimensional inputs whose elements are organized within two or more dimensions. The organization of the ANN along these dimensions may be related to some structure in the input structure (e.g., as relative location within the two-dimensional space of an image can be related to similarity between pixels of the image).

In example embodiments, a CNN includes at least one two-dimensional (or higher-dimensional) filter that is applied to an input; the filtered input is then applied to neurons of the CNN (e.g., of a convolutional layer of the CNN). The convolution of such a filter and an input could represent the color values of a pixel or a group of pixels from the input, in embodiments where the input is an image. A set of neurons of a CNN could receive respective inputs that are determined by applying the same filter to an input. Additionally or alternatively, a set of neurons of a CNN could be associated with respective different filters and could receive respective inputs that are determined by applying the respective filter to the input. Such filters could be trained during training of the CNN or could be pre-specified. For example, such filters could represent wavelet filters, center-surround filters, biologically-inspired filter kernels (e.g., from studies of animal visual processing receptive fields), or some other pre-specified filter patterns.

A CNN or other variety of ANN could include multiple convolutional layers (e.g., corresponding to respective different filters and/or features), pooling layers, rectification layers, fully connected layers, or other types of layers. Convolutional layers of a CNN represent convolution of an input image, or of some other input (e.g., of a filtered, downsampled, or otherwise-processed version of an input image), with a filter. Pooling layers of a CNN apply non-linear downsampling to higher layers of the CNN, e.g., by applying a maximum, average, L2-norm, or other pooling function to a subset of neurons, outputs, or other features of the higher layer(s) of the CNN. Rectification layers of a CNN apply a rectifying nonlinear function (e.g., a non-saturating activation function, a sigmoid function) to outputs of a higher layer. Fully connected layers of a CNN receive inputs from many or all of the neurons in one or more higher layers of the CNN. The outputs of neurons of one or more fully connected layers (e.g., a final layer of an ANN or CNN) could be used to determine information about areas of an input image (e.g., for each of the pixels of an input image) or for the image as a whole.

Neurons in a CNN can be organized according to corresponding dimensions of the input. For example, where the input is an image (a two-dimensional input, or a three-dimensional input where the color channels of the image are arranged along a third dimension), neurons of the CNN (e.g., of an input layer of the CNN, of a pooling layer of the CNN) could correspond to locations in the two-dimensional input image. Connections between neurons and/or filters in different layers of the CNN could be related to such locations. For example, a neuron in a convolutional layer of the CNN could receive an input that is based on a convolution of a filter with a portion of the input image, or with a portion of some other layer of the CNN, that is at a location proximate to the location of the convolutional-layer neuron. In another example, a neuron in a pooling layer of the CNN could receive inputs from neurons, in a layer higher than the pooling layer (e.g., in a convolutional layer, in a higher pooling layer), that have locations that are proximate to the location of the pooling-layer neuron.

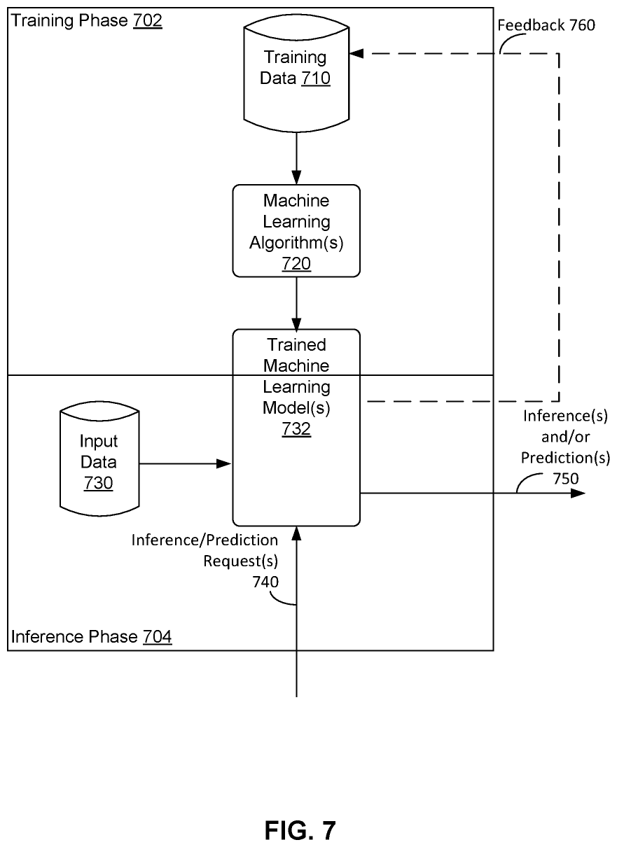

FIG. 7 shows diagram 700 illustrating a training phase 702 and an inference phase 704 of trained machine learning model(s) 732, in accordance with example embodiments. Some machine learning techniques involve training one or more machine learning algorithms, on an input set of training data to recognize patterns in the training data and provide output inferences and/or predictions about (patterns in the) training data. Such output could take the form of filtered or otherwise modified versions of the input, e.g., an input image could be modified by the machine learning model to appear as though foreground content is in-focus while background content is out of focus. The resulting trained machine learning algorithm can be termed as a trained machine learning model. For example, FIG. 7 shows training phase 702 where one or more machine learning algorithms 720 are being trained on training data 710 to become trained machine learning model 732. Then, during inference phase 704, trained machine learning model 732 can receive input data 730 and one or more inference/prediction requests 740 (perhaps as part of input data 730) and responsively provide as an output one or more inferences and/or predictions 750.

As such, trained machine learning model(s) 732 can include one or more models of one or more machine learning algorithms 720. Machine learning algorithm(s) 720 may include, but are not limited to: an artificial neural network (e.g., a herein-described convolutional neural networks, a recurrent neural network, a Bayesian network, a hidden Markov model, a Markov decision process, a logistic regression function, a support vector machine, a suitable statistical machine learning algorithm, and/or a heuristic machine learning system), a support vector machine, a regression tree, an ensemble of regression trees (also referred to as a regression forest), a decision tree, an ensemble of decision trees (also referred to as a decision forest), or some other machine learning model architecture or combination of architectures. Machine learning algorithm(s) 720 may be supervised or unsupervised, and may implement any suitable combination of online and offline learning.

In some examples, machine learning algorithm(s) 720 and/or trained machine learning model(s) 732 can be accelerated using on-device coprocessors, such as graphic processing units (GPUs), tensor processing units (TPUs), digital signal processors (DSPs), and/or application specific integrated circuits (ASICs). Such on-device coprocessors can be used to speed up machine learning algorithm(s) 720 and/or trained machine learning model(s) 732. In some examples, trained machine learning model(s) 732 can be trained, reside and execute to provide inferences on a particular computing device, and/or otherwise can make inferences for the particular computing device.

During training phase 702, machine learning algorithm(s) 720 can be trained by providing at least training data 710 as training input using unsupervised, supervised, semi-supervised, and/or reinforcement learning techniques. Unsupervised learning involves providing a portion (or all) of training data 710 to machine learning algorithm(s) 720 and machine learning algorithm(s) 720 determining one or more output inferences based on the provided portion (or all) of training data 710. Supervised learning involves providing a portion of training data 710 to machine learning algorithm(s) 720, with machine learning algorithm(s) 720 determining one or more output inferences based on the provided portion of training data 710, and the output inference(s) are either accepted or corrected based on correct results associated with training data 710. In some examples, supervised learning of machine learning algorithm(s) 720 can be governed by a set of rules and/or a set of labels for the training input, and the set of rules and/or set of labels may be used to correct inferences of machine learning algorithm(s) 720.

Semi-supervised learning involves having correct results for part, but not all, of training data 710. During semi-supervised learning, supervised learning is used for a portion of training data 710 having correct results, and unsupervised learning is used for a portion of training data 710 not having correct results. Reinforcement learning involves machine learning algorithm(s) 720 receiving a reward signal regarding a prior inference, where the reward signal can be a numerical value. During reinforcement learning, machine learning algorithm(s) 720 can output an inference and receive a reward signal in response, where machine learning algorithm(s) 720 are configured to try to maximize the numerical value of the reward signal. In some examples, reinforcement learning also utilizes a value function that provides a numerical value representing an expected total of the numerical values provided by the reward signal over time. In some examples, machine learning algorithm(s) 720 and/or trained machine learning model(s) 732 can be trained using other machine learning techniques, including but not limited to, incremental learning and curriculum learning.

In some examples, machine learning algorithm(s) 720 and/or trained machine learning model(s) 732 can use transfer learning techniques. For example, transfer learning techniques can involve trained machine learning model(s) 732 being pre-trained on one set of data and additionally trained using training data 710. More particularly, machine learning algorithm(s) 720 can be pre-trained on data from one or more computing devices and a resulting trained machine learning model provided to computing device CD1, where CD1 is intended to execute the trained machine learning model during inference phase 704. Then, during training phase 702, the pre-trained machine learning model can be additionally trained using training data 710, where training data 710 can be derived from kernel and non-kernel data of computing device CD1. This further training of the machine learning algorithm(s) 720 and/or the pre-trained machine learning model using training data 710 of CD1's data can be performed using either supervised or unsupervised learning. Once machine learning algorithm(s) 720 and/or the pre-trained machine learning model has been trained on at least training data 710, training phase 702 can be completed. The trained resulting machine learning model can be utilized as at least one of trained machine learning model(s) 732.

In particular, once training phase 702 has been completed, trained machine learning model(s) 732 can be provided to a computing device, if not already on the computing device. Inference phase 704 can begin after trained machine learning model(s) 732 are provided to computing device CD1.

During inference phase 704, trained machine learning model(s) 732 can receive input data 730 and generate and output one or more corresponding inferences and/or predictions 750 about input data 730. As such, input data 730 can be used as an input to trained machine learning model(s) 732 for providing corresponding inference(s) and/or prediction(s) 750 to kernel components and non-kernel components. For example, trained machine learning model(s) 732 can generate inference(s) and/or prediction(s) 750 in response to one or more inference/prediction requests 740. In some examples, trained machine learning model(s) 732 can be executed by a portion of other software. For example, trained machine learning model(s) 732 can be executed by an inference or prediction daemon to be readily available to provide inferences and/or predictions upon request. Input data 730 can include data from computing device CD1 executing trained machine learning model(s) 732 and/or input data from one or more computing devices other than CD1.

Input data 730 can include a collection of images provided by one or more sources. The collection of images can include video frames, images resident on computing device CD1, and/or other images. Other types of input data are possible as well.

Inference(s) and/or prediction(s) 750 can include output images, output intermediate images, output vectors embedded in a multi-dimensional space, numerical values, and/or other output data produced by trained machine learning model(s) 732 operating on input data 730 (and training data 710). In some examples, trained machine learning model(s) 732 can use output inference(s) and/or prediction(s) 750 as input feedback 760. Trained machine learning model(s) 732 can also rely on past inferences as inputs for generating new inferences.

### V. Example Embodiments and Experimental Results

Approximately 837 million chest radiographs are obtained annually worldwide for detecting, diagnosing, and managing cardiothoracic conditions; chest radiography is also considerably more accessible than CT in many parts of the world. Serious effort has been invested into developing deep-learning models to detect chest radiography abnormalities. However, core challenges for model development include the need for extremely large, labeled training data sets and the ability to generalize to different populations and institutions.

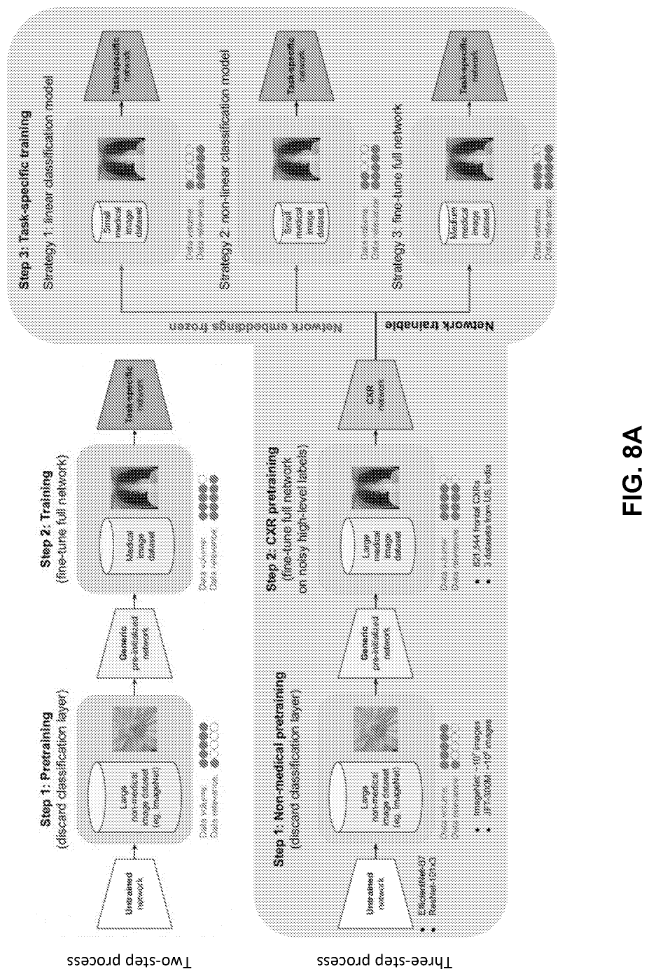

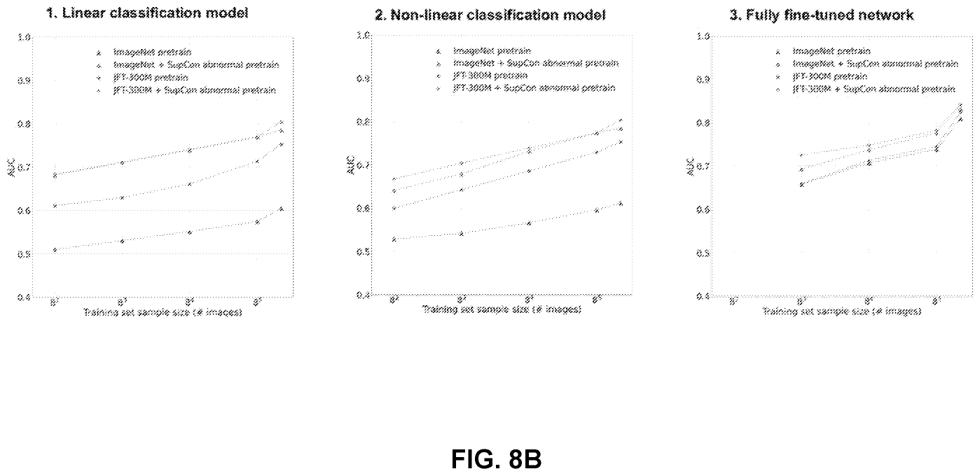

Transfer learning, a machine-learning approach that repurposes a model trained on one task for a different but related task, may reduce the need for large data sets. For example, a transfer learning workflow can involve first pre-training a deep-learning model on a generic source task (often using large nonmedical data sets) and then refining the model on a specific target medical task (using a medical data set). An example of such a process is depicted as the “Two-step process” in FIG. 8A. Although transfer learning is more effective when the source and target tasks are similar (e.g., both medical), this would typically require tens of thousands of labeled medical images. Fortunately, this obstacle of lacking medical labels can be partially overcome by using self-supervised machine-learning techniques that can make use of unlabeled data.

Example systems and methods are described herein to facilitate modeling chest radiograph-specific tasks through a three-step training setup: generic image pretraining, chest radiograph-specific pre-training, and task-specific training. The first step uses large nonmedical image data sets for pre-training. The second step uses chest radiography data sets (or other commonly available medical image data) with scalable albeit noisy labels of abnormality from natural language processing of radiology reports, in combination with a supervised contrastive (SupCon) learning approach to build a chest radiography network. This chest radiography network converts chest radiographs into information-rich numerical vectors (“embeddings,” which, depending on the specific network, may be hundreds to thousands of entries in length) that can be used to more easily train models for specific medical prediction tasks (e.g., image finding, clinical condition, or patient outcome).

This approach was experimentally evaluated by measuring the data versus performance tradeoff under three training scenarios: (a) a linear model applied to frozen embeddings, (b) a nonlinear model applied to frozen embeddings, and (c) a nonlinear model produced by fine-tuning the entire network. The results provided herein suggest that performant models can be trained from as few as 10-100 examples. To accelerate chest radiography modeling efforts with low data and computational requirements, the functionality of such a trained model can be provided as a service (e.g., via a cloud computing service or other online service) along with scripts to train linear and nonlinear classifiers on top of the outputs of the generic model.

### Materials and Methods

### Experiment Design

Whether the three-step training setup described herein improves the final performance of prediction tasks was evaluated. The baseline used for comparison was a two-step training setup that started with pre-training from a large nonmedical data set to produce a generic pre-initialized network (“Two-step process” of FIG. 8A). The three-step training setup included an additional pre-training step that used a large chest radiography data set with radiology report-derived labels (‘abnormal’ or ‘normal’) to create a chest radiography network (“Three-step process” of FIG. 8A). The last step in both setups was task-specific training, with the task being an image finding, clinical condition, or patient outcome. Note that the chest radiography network pre-training used noisy radiology report-derived labels, while the medical task-specific training as well as task-specific evaluations used clean labels from radiologist image reviews, molecular testing, or clinical outcomes. How well the embeddings (from the fully generic network or the chest radiography network) generated by means of these two-step and three-step training setups performed was assessed in linear classification, nonlinear classification, and as a starting point for fine-tuning of the full network. The use of different-sized training data sets was simulated by subsampling the training data sets.

In these experiments, the SupCon approach was used to build two generic pre-initialized networks from two well-known nonmedical image data sets: ImageNet, a data set containing 14 million natural images and commonly used to initialize machine-learning models for other applications (with approximately 107 images), and JFT-300M, a larger data set consisting of 300 million weakly labeled natural images used in this work as an alternative to ImageNet initialization (with approximately 108 images). For each data set, a different network architecture (a documented design of how the network's layers and connections are arranged) was used—the small modern neural network architecture EfficientNet-B7 for the ImageNet data set and the larger architectures ResNet-101x3 and ResNet-152x4 for the larger JFT-300M data set. Henceforth, experiments described as using the data sets “ImageNet” and “JFT-300M” will also describe their associated architectures, unless otherwise specified.

### Chest Radiography Data Sets

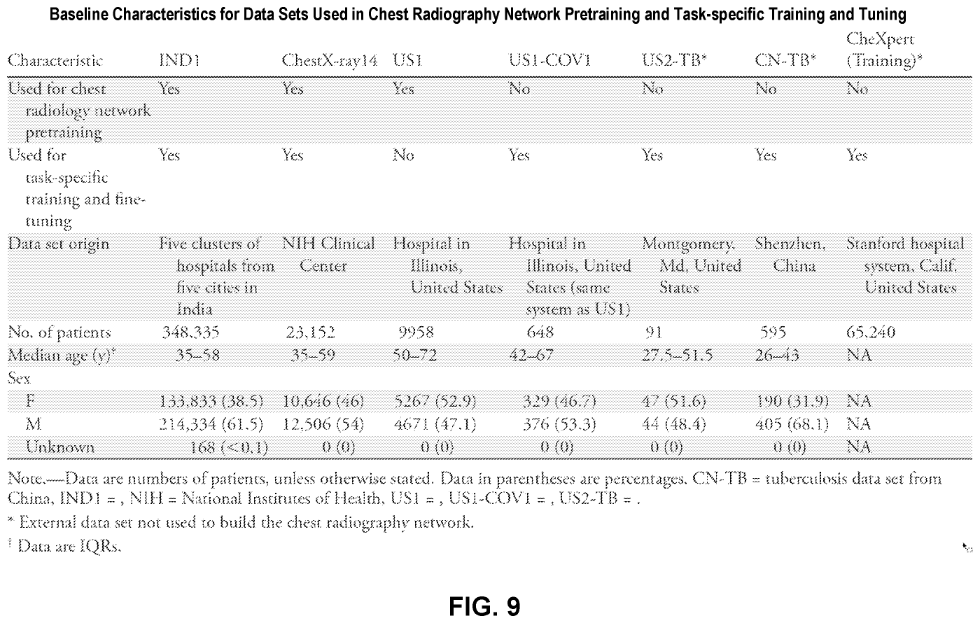

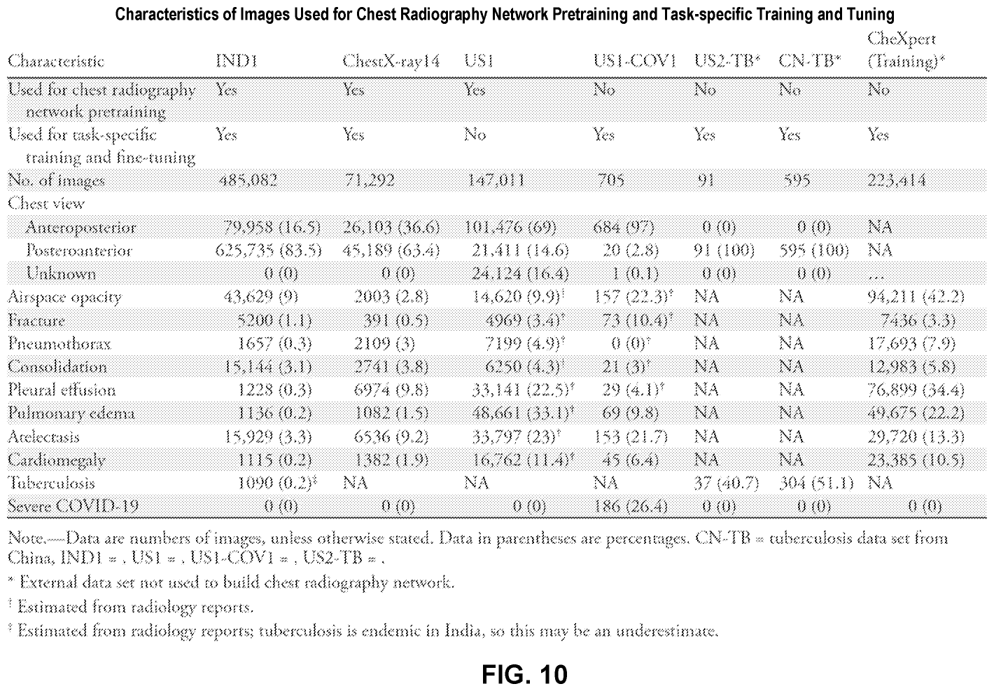

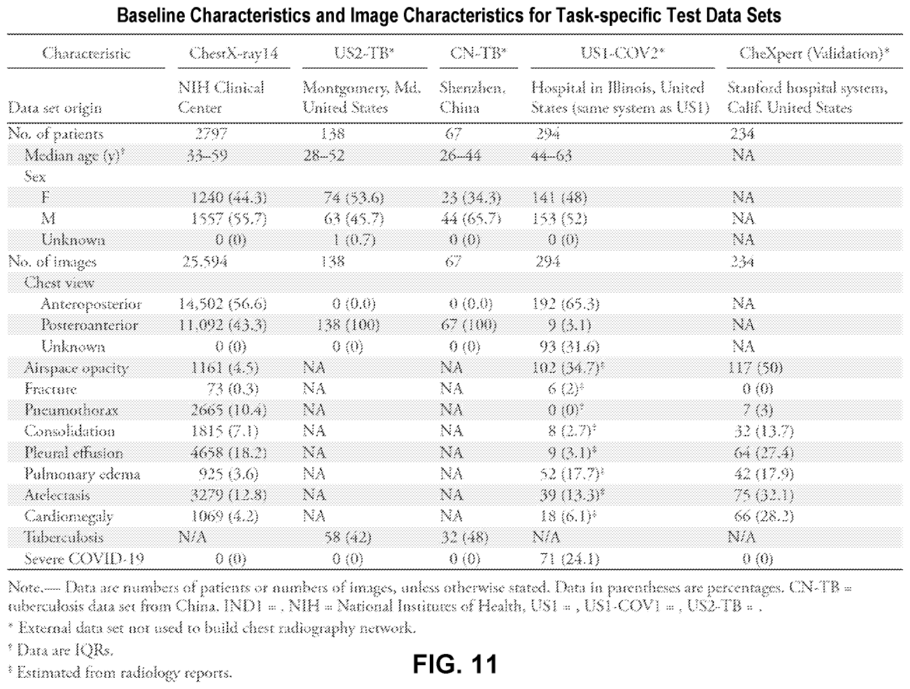

The medical data sets used are detailed in FIGS. 9-11. The chest radiographs used to produce the chest radiography network included more than 700,000 images across five hospitals in five cities in India (hereafter, IND1 data set), the ChestX-ray14 data set, and a hospital from Illinois in the United States (hereafter, US1 data set). The data sets for training the task-specific models included IND1, ChestX-ray14, and CheXpert for the general chest radiography findings. The tuberculosis setup attempted the classification both ways-training on tuberculosis data sets from the United States (hereafter, US2-TB) and evaluating on tuberculosis data sets from China (hereafter, CN-TB), and training on CN-TB and evaluating on US-TB. The COVID-19 prediction task involved training on COVID-19 data sets from the United States (hereafter, US1-COV1) and evaluating on a separate site, US2-COV2. Independent external validation test data sets include US2-TB2, CN-TB, and CheXpert.

### Findings Studied

Experiments were conducted across multiple findings and/or diseases and clinical outcomes (outlined in FIGS. 9-11). These included multiple imaging findings found in a general clinical settings (six findings in the ChestX-ray14 data set and five findings in the CheXpert data set), microbiologically confirmed tuberculosis in two publicly available data sets from Montgomery County in the United States (US2-TB) and Shenzhen, China (CN-TB) (which have both tuberculosis-positive and -negative chest radiography networks), and five important clinical end points (four individual end points and one composite end point). Ground truth tuberculosis status was provided with the two public data sets (US2-TB2, CN-TB).

### Chest Radiography Network Pre-Training by Means of SupCon

The second pre-training step produces a chest radiography network using SupCon. SupCon builds on the self-supervised learning technique, a simple framework for contrastive learning of visual representations, or SimCLR, which is designed to encourage the network to learn a good representation from unlabeled examples by leveraging the idea that crops of the same image (A) are more similar than crops from different source images (A and B). SupCon extends this by leveraging the idea that images of the same class (e.g., class ‘0’) are more similar than images from different classes (e.g., class ‘0’ and class ‘1’). Here, this concept was used to apply noisy labels as to whether a chest radiograph contains abnormal findings or not. These noisy labels were extracted from natural language processing of radiologist reports in combination with more specific labels (e.g., ChestX-ray14). This chest radiography network converts chest radiographs into high-dimensional numerical vectors (embeddings). Such an embedding of an image is a numerical representation that can represent the information contained in that image, such that a simple model can use this “summary” embedding as input for prediction tasks. Because such simple models (e.g., small linear or nonlinear models) may be smaller than the full network, they often require much less data for training.

### Task-Specific Model Training

Three types of task-specific model development were investigated: (a) a linear model applied to frozen embeddings, (b) a nonlinear model applied to frozen embeddings, and (c) a model produced by fine-tuning the entire network (including the embeddings). The first two models use static frozen embeddings created by using the chest radiography network to transform each chest radiograph in the task-specific data sets (train, tune, test) to an embedding. The linear model consisted of training a single-layer linear probe, which makes one prediction for each embedding. The nonlinear model is similar but includes a multilayer perceptron instead of a single layer. The third approach began from the same pre-trained chest radiography network but added a custom classification layer and subsequently fine-tuned the entire network for each prediction task, which enabled the embeddings to be refined for the specific prediction tasks.

### Evaluation

To evaluate the model's sensitivity to data set size and ability to generalize across multiple tasks, the training data set was subsampled to five sizes spanning a logarithmic scale of 64, 512, 4096, 32,768, and 68,801 or 674,533 samples. The maximum training set sample size was based on the availability of labels across multiple data sets; for example, airspace opacity, fracture, and pneumothorax were available in both ChestX-ray14 and IND1 (and subsampled up to 674,533), whereas consolidation, pleural effusion, and pulmonary edema were only available in ChestXray14 (and subsampled up to 68,801). Sampling was stratified to maintain the positive class ratio and to ensure that all subsamples contained both classes. Note that the full network refinement approach was not attempted for the smallest data set size (n=64) due to the small size. Because all prediction tasks were binary, the area under the receiver operating characteristic curve (AUC) was used for all evaluations. Comparator radiologists for tuberculosis were India-based consultants with radiology certifications who had experience in reading tuberculosis images.

### Visualizing the Embeddings at Each Step

To better understand how the embeddings change at each step, t-distributed Stochastic Neighbor Embedding, or t-SNE, a technique for visualizing high-dimensional data was used. Although the units are not always quantitatively interpretable, the qualitative observation of how data points are distributed can give an idea of how examples (chest radiographs) from different classes are spread out in the high-dimensional embedding space. This method was used to observe the separation between positive and negative labels at each of the three steps of the training process (generic network, chest radiography network, task-specific network).

### Results

The experimental results provided herein assess whether adding an additional pre-training step to produce a chest radiography network improves the final performance for prediction tasks and under what data-set-size regimes this occurs.

### General Clinical Findings (ChestX-Ray14)

In the ChestX-ray14 data set, on average across six findings, SupCon substantially and consistently improved accuracy of models developed across a range of training data set sizes for both frozen embedding scenarios (linear classification and nonlinear classification). When the entire network was fine-tuned, an improvement was also observed, but the baselines were substantially higher, and the gains were smaller (see FIG. 8B). These trends were generally consistent for both ImageNet and JFT-300M, although with a much higher baseline performance for the JFT-300M (which has more parameters and was pretrained on a larger data set; see FIG. 8B). With the application of SupCon, the performance converged (see FIG. 8B).

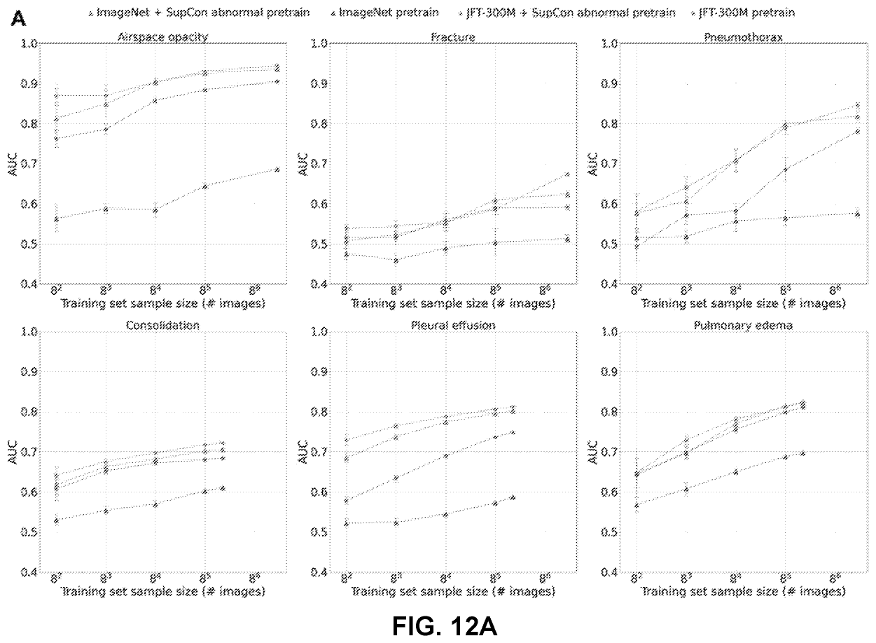

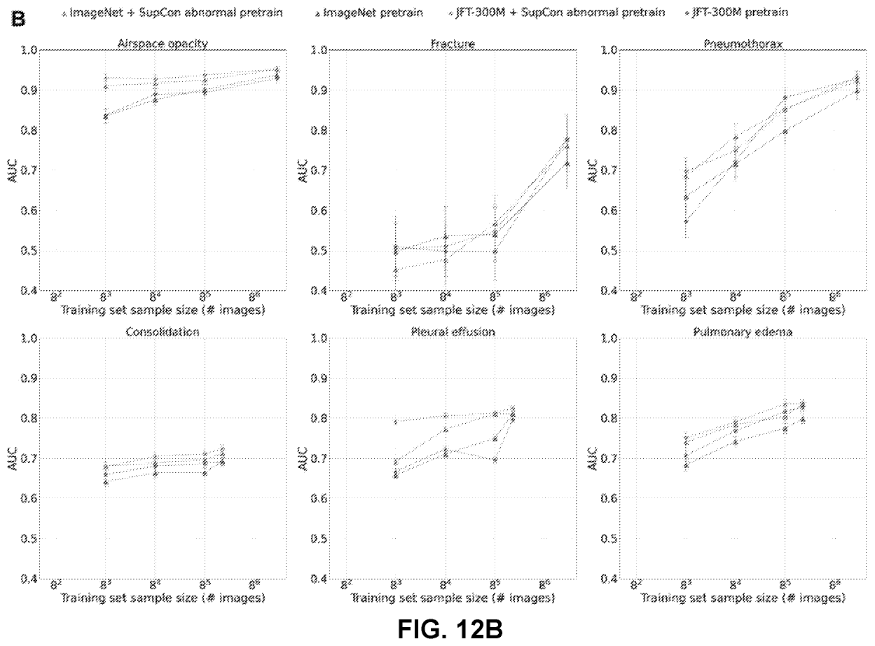

FIG. 8B depicts the results averaged across multiple tasks (airspace opacity, fracture, pneumothorax, consolidation, pleural effusion, and pulmonary edema) on the ChestX-ray14 data set; see FIGS. 12A-B for the same analysis per task. The final training set size is the largest size available for each task (at least 68,801) and differs based on task. AUC=area under the receiver operating characteristic curve, ImageNet=data set containing natural images, JFT-300M=larger data set containing natural images.

When radiologists assessed the individual findings for the nonlinear cap network (see FIG. 12A) and for the fine-tuned network (see FIG. 12B), the observations remained generally similar for five findings, although with widely varying performances across findings. Pre-training with SupCon demonstrated statistically superior performance compared with not using SupCon (P<1e-5). The exception to the other five findings was fractures, for which none of the approaches tested performed particularly well at low training-set sizes.

FIGS. 12A-B show the effect of using the chest radiography network developed using the three-step training setup for task-specific prediction on ChestX-ray14 data set. Results are from FIG. 8B and were sectioned on a per-finding basis. FIG. 12A depicts results with nonlinear model using frozen embeddings. AUC=area under the receiver operating characteristic. FIG. 12B depicts results with fine-tuning the full network. Results for the linear model are similar though generally slightly lower than that for the nonlinear models. ImageNet=data set containing natural images, JFT-300M=larger data set containing natural images.

### General Clinical Findings (CheXpert)

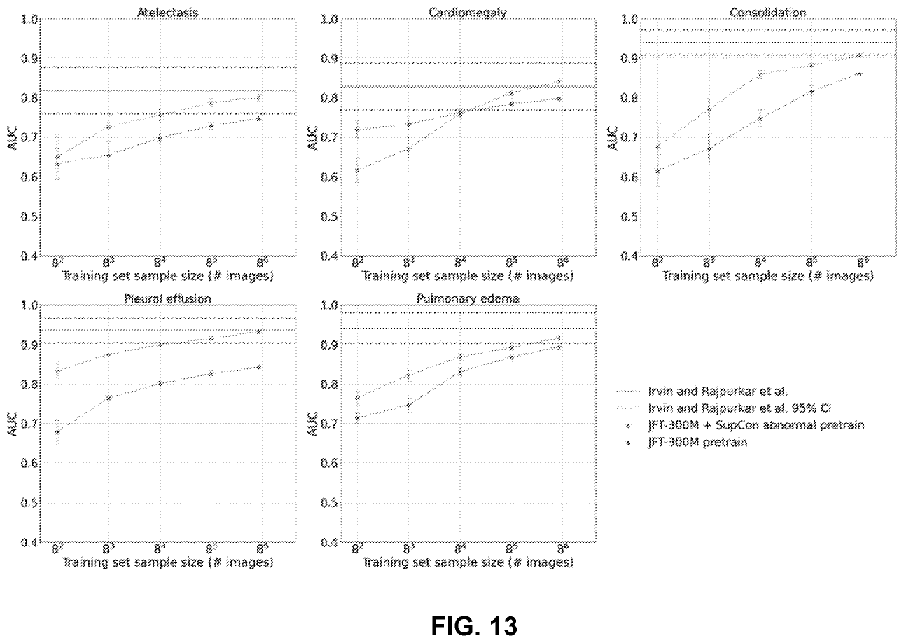

SupCon was also benchmarked on the publicly available CheXpert data set (an external data set not used for pre-training), with a slightly different set of five findings (see FIG. 13) and a larger architecture for the JFT-300M pretraining, ResNet-152x4 (which generally showed better performance than ResNet-101x3). The observations were generally similar to those seen in ChestXray14 for four of five findings. The exception was cardiomegaly, where the use of SupCon actually was associated with a lower performance at the smallest data set sizes (n=64 and 512), but which reversed to similarly show higher performance with SupCon (n=4096, 32,768, and 224,316). When these results were compared to that of the original CheXpert model, SupCon enabled comparable performance (as assessed by performance within the CIs) at 84 images for atelectasis, cardiomegaly, and pleural effusion and 85 images for pulmonary edema.

FIG. 13 shows the effect of using the chest radiography network from the three-step training setup with nonlinear classifiers on task-specific findings in the CheXpert data set. Solid and dotted horizontal lines indicate performance of the original CheXpert model on all available training data (224,000 images); area under the receiver operating characteristic curves (AUCs) for the nonlinear models trained on 1% and 10% of the training set (data points at 84 and 85 above) approached published performance of the original CheXpert model for atelectasis, cardiomegaly, pleural effusion, and pulmonary edema.

### Tuberculosis

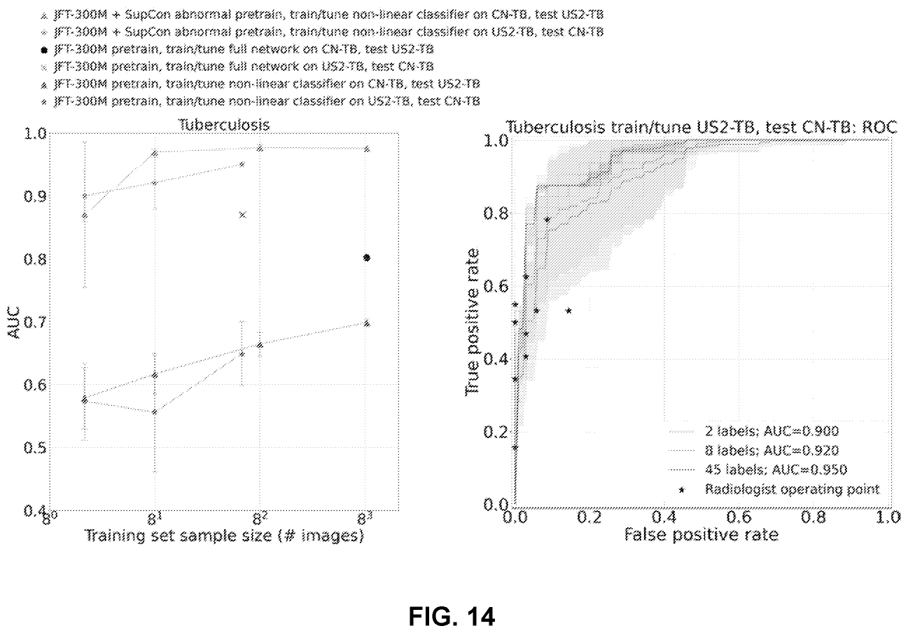

When evaluated for its ability to develop tuberculosis-detection models (using two external data sets), SupCon showed similar trends as general clinical findings on CheXpert data set, although the maximum data set sizes were smaller, and the delta was more striking (see FIG. 14). This held true whether training on US2-TB and testing on CN-TB or vice versa. The naïve approach of fine-tuning the entire network on tuberculosis labels (circle and cross) performs less well than the nonlinear models on top of frozen embeddings (see FIG. 14, left pane). The resultant models (all 10 trained on random subsamples of the training data) attained non-inferiority to India-based radiologists in detecting tuberculosis when using just 45 training images, and the AUC reached 0.92 with eight training images (see FIG. 14, right pane). At all training set sizes (including two and eight images), the model trained on US2-TB had a receiver operating curve comparable to that of radiologists (40%-60% sensitivity at near-perfect specificity) in the CN-TB data set.

FIG. 14 shows the effect of using the chest radiology network from the three-step training setup with nonlinear classifiers for tuberculosis detection. Radiologist non-inferiority was achieved with orders of magnitude of fewer training examples; a nonlinear model trained on 45 images from the tuberculosis data set from the United States (US2-TB) was non-inferior to 10 India-based radiologists (P<1e-5) on the external validation data set (tuberculosis data set from China [CN-TB]). The ‘x’ shows fine-tuning the entire network on US2-TB with testing on CN-TB (area under the receiver operating characteristic curve [AUC]=0.87). The circle shows fine-tuning the entire network on CN-TB with testing on US2-TB (AUC=0.80). Lightly shaded areas (right) represent the 95% CIs of models trained on different random subsets of US2-TB, with the dark lines corresponding to the mean. India-based radiologist consultants had an average of 6 years of experience (range, 3-9 years).

### COVID-19 Clinical Outcomes

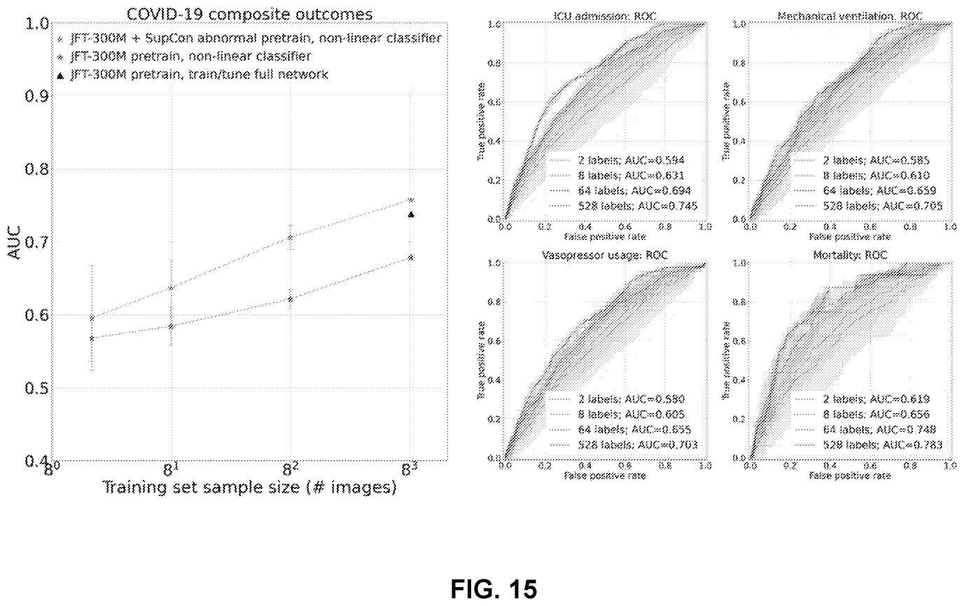

The ability of the models described herein to predict four key clinical outcomes for patients with COVID-19, as well as a composite end point encompassing all four outcomes, was also assessed. Despite the relatively small training data set containing 12-173 outcomes, the performance of a nonlinear model (JFT-300M) rose rapidly with increasing sample size, attaining an AUC of 0.75 with use of just 528 examples (see FIG. 15). The nonlinear classifier trained on frozen embeddings outperformed a model that was fine-tuned on the entire data set.

FIG. 15 shows the effect of the use of the chest radiography network from the three-step training setup with nonlinear classifiers to predict COVID-19 outcomes. The performance increases rapidly despite having only hundreds of training examples (with a quarter experiencing the outcomes). A nonlinear classifier trained (with a fraction of the compute resources) on frozen embeddings of the whole data set (528 images) also outperformed a network pre-trained on JFT-300M and fully fine-tuned on the whole data set (triangle, area under the receiver operating characteristic curve [AUC]=0.74). Lightly shaded areas (right) represent the 95% CIs of models trained on different random subsets of the tuberculosis data set from the United States, with the dark lines corresponding to the mean. ICU=intensive care unit.

### Visualizing the Embeddings at Each Step

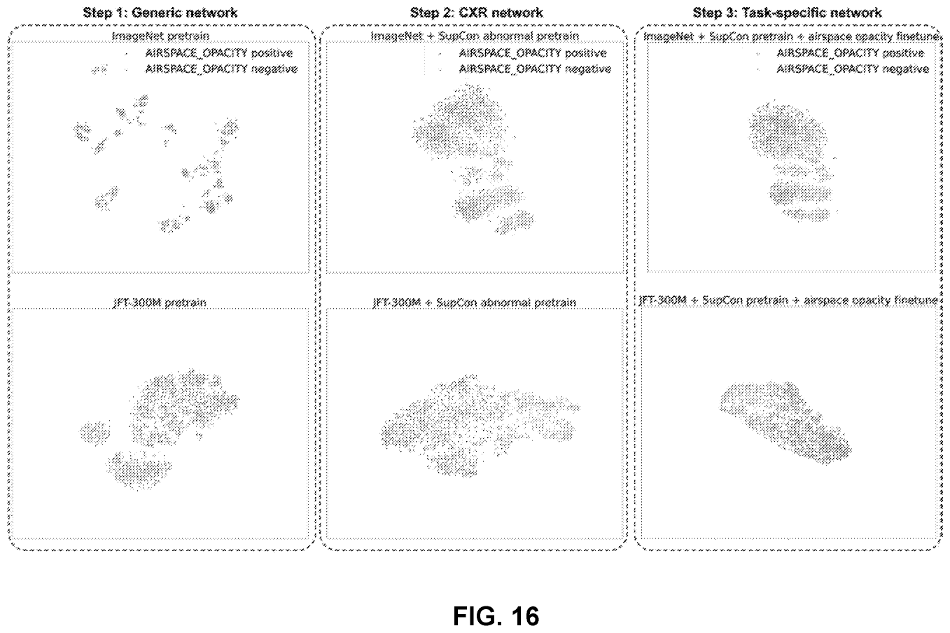

The example case of airspace opacity is used (in FIG. 16) to illustrate that the SupCon embeddings from the chest radiography network produced a better visual separation than the generic pre-trained network and a visually comparable separation to the fully fine-tuned task-specific network.

FIG. 16 depicts t-Distributed Stochastic Neighbor Embedding (t-SNE) visualizations of the embeddings at each step in the three-step training setup. The supervised contrastive (SupCon) embeddings produced a better visual separation of the classes (middle) than the generic pretrained network (left) and as good a separation as a fully fine-tuned network (right). Note that this visualization technique leverages highly nonlinear axes, so neither axis can be assigned readily interpretable units. CXR=chest radiography.

### DISCUSSION

The experimental results provided herein assess a three-step training setup that involved generating a chest radiography network to accelerate the building of task-specific deep-learning models. The primary results are: (a) a simple natural language processing of radiology reports can scalably generate weak labels for the supervised contrastive learning approach used for building the chest radiography network; (b) in small data regimens the resultant embeddings can improve the task-specific classification performance substantially, by as much as an absolute area under the receiver operating characteristic curve of 0.1-0.2; (c) similar performance is obtainable with three- to 688-fold less data; and (d) the gains were less prominent in the large data regime.

When using the chest radiography network described herein in a frozen manner and training simple linear and nonlinear models thereon, performance increased when using SupCon on most task-specific predictions in a data set that was also used for pretraining (ChestX-ray14), as well as an external data set not used for pretraining (CheXpert). This improvement generalized to identification of medical disease not labeled in pre-training (tuberculosis) and to prediction of downstream clinical outcomes (COVID-19 clinical outcomes). While the benefits seen for these two tasks were large, the maximum training data set size was only in the hundreds. Additionally, when data sets were small, as in the case of the tuberculosis data sets, fine-tuning the entire network was less robust than the use of nonlinear models on top of frozen embeddings. Finally, the choice of architecture and generic pre-training data set (ImageNet and JFT-300M) made less of a difference in performance than the gains from using SupCon; both architectures performed similarly when combined with SupCon (see FIGS. 8B, 12A-B).

Although the observation that SupCon substantially improved performance was generally highly consistent across architectures, data sets, and tasks, there were two tasks that showed less robust improvements: cardiomegaly and fracture. While the trendline for SupCon had lower performance with low sample sizes (see FIG. 13), it improved more rapidly and surpassed the control at around 84 examples.

The results provided herein show that the chest radiography network described herein can perform well with as few as hundreds of task-specific training examples. By using the COVID-19 pandemic as an example, as the affected demographic and severity of illness changed over time (perhaps affected by virus variants, improved medical interventions, availability and use of vaccines, and changing viral transmission patterns), model updates may be more easily achieved by means of these data-efficient techniques. This chest radiography network may also be useful in the study of less common diseases, which is also often limited by small data. Yet another area where the approach presented may be of value lies in institutions and teams desiring to develop and study custom models for their local task of interest and patient population and, thus, operating in the regime of small data.

The results provided herein show that pre-training on scalably extractable noisy labels can be used to provide generalizable embeddings that substantially improve predictive performance across a wide range of data sets and prediction tasks with as few as tens of examples. This unlocks the ability to rapidly train chest radiography models on smaller data sets or when data are scarce.

### VI. Conclusion

The particular arrangements shown in the Figures should not be viewed as limiting. It should be understood that other embodiments may include more or less of each element shown in a given Figure. Further, some of the illustrated elements may be combined or omitted. Yet further, an exemplary embodiment may include elements that are not illustrated in the Figures.

Additionally, while various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims. Other embodiments may be utilized, and other changes may be made, without departing from the spirit or scope of the subject matter presented herein. It will be readily understood that the aspects of the present disclosure, as generally described herein, and illustrated in the figures, can be arranged, substituted, combined, separated, and designed in a wide variety of different configurations, all of which are contemplated herein.

1. A computer-implemented method comprising:
receiving, by a first computing system, a specific medical training data set that includes a plurality of medical diagnostic images that are associated with a specific condition or diagnosis and a plurality of diagnostic labels associated therewith;
transmitting, by the first computing system to a second computing system, the plurality of medical diagnostic images;
receiving, by the first computing system from the second computing system, a plurality of output vectors, wherein each output vector of the plurality of output vectors represents an embedding of a respective one of the plurality of medical diagnostic images into a multi-dimensional embedding space; and
training, by the first computing system using the plurality of diagnostic labels and the plurality of output vectors, a trained machine learning model is configured to receive a target output vector that represents an embedding of a target input image into the multi-dimensional embedding space and to output, based on the target output vector, an indication of at least one of a presence, degree of severity, or type of the specific condition or diagnosis.

2. The computer-implemented method of claim 1, further comprising:
transmitting, by the first computing system to the second computing system, a target medical diagnostic image;
receiving, by the first computing system from the second computing system, an indication of a target output vector that represents an embedding of the target medical diagnostic image into the multi-dimensional embedding space; and
applying, by the first computing system, the target output vector to the trained machine learning model.

3-4. (canceled)

5. A computer-implemented method comprising:
transmitting, by a first computing system to a second computing system, a target medical diagnostic image;
receiving, by the first computing system from the second computing system, a target output vector that represents an embedding of the target medical diagnostic image into a multi-dimensional embedding space; and
applying, by the first computing system, the target output vector to a trained machine learning model to generate a target indication of at least one of a presence, degree of severity, or type of the specific condition or diagnosis represented in the target medical diagnostic image.

6-7. (canceled)

8. The computer-implemented method of claim 5, further comprising:
receiving, by the first computing system from the second computing system, an indication of the trained machine learning model.

9. A computer-implemented method comprising:
receiving a first trained machine learning model, wherein the first trained machine learning model is configured to receive an image as an input and to output, based on the input image, an output vector that represents an embedding of the input image into a first multi-dimensional embedding space;
generating a second trained machine learning model by using a generic medical training data set to further train the first trained machine learning model, wherein the generic medical training data set includes a plurality of medical diagnostic images and a plurality of diagnostic labels associated therewith, wherein the second trained machine learning model is configured to receive an image as an input and to output, based on the input image, an output vector that represents an embedding of the input image into a second multi-dimensional embedding space; and
using a specific medical training data set and the second trained machine learning model, generating a third trained machine learning model, wherein the specific medical training data set includes a plurality of medical diagnostic images that are associated with a specific condition or diagnosis and a plurality of diagnostic labels associated therewith, wherein the third trained machine learning model is configured to receive an image as an input and to output, based on the input image, an output that is representative of a property or presence of the specific condition or diagnosis.

10. The computer-implemented method of claim 9, wherein generating the third trained machine learning model comprises using the specific medical training data set to further train the second trained machine learning model, thereby generating the third trained machine learning model from the second trained machine learning model, and wherein the third trained machine learning model is configured to receive an image as an input and to output, based on the input image, an output vector that represents an embedding of the input image into a third multi-dimensional embedding space.

11. (canceled)

12. The computer-implemented method of claim 9, wherein the third trained machine learning model comprises the second trained machine learning model and a fourth trained machine learning model, wherein the fourth trained machine learning model is configured to receive an output vector from the second trained machine learning model that represents an embedding of an input image into the second multi-dimensional embedding space and to output, based on the output vector from the second trained machine learning model, an indication of at least one of a presence, degree of severity, or type of the specific condition or diagnosis; and wherein the method further comprises:
transmitting, from a first computing system to a second computing system, an indication of the fourth trained machine learning model;
receiving, by the first computing system from the second computing system, a target medical diagnostic image;
applying, by the first computing system, the target medical diagnostic image to the second trained machine learning model to generate a target output vector that represents an embedding of the target medical diagnostic image into the second multi-dimensional embedding space; and
transmitting, by the first computing system to the second computing system, an indication of the target output vector.

13. The computer-implemented method of claim 9, wherein the third trained machine learning model comprises the second trained machine learning model and a fourth trained machine learning model, wherein the fourth trained machine learning model is configured to receive an output vector from the second trained machine learning model that represents an embedding of an input image into the second multi-dimensional embedding space and to output, based on the output vector from the second trained machine learning model, an indication of at least one of a presence, degree of severity, or type of the specific condition or diagnosis; and wherein the method further comprises:
receiving, by the first computing system from the second computing system, a target medical diagnostic image;
applying, by the first computing system, the target medical diagnostic image to the second trained machine learning model to generate a target output vector that represents an embedding of the target medical diagnostic image into the second multi-dimensional embedding space;
applying, by the first computing system, the target output vector to the fourth trained machine learning model to generate a target indication of at least one of a presence, degree of severity, or type of the specific condition or diagnosis represented in the target medical diagnostic image; and
transmitting, by the first computing system to the second computing system, the target indication.

14-16. (canceled)

17. The computer-implemented method of claim 9, wherein the diagnostic labels of the plurality of diagnostic labels indicate whether their associated medical diagnostic images are normal or abnormal.

18. The computer-implemented method of claim 17, further comprising:
generating the plurality of diagnostic labels based on medical records associated with the plurality of medical diagnostic images, wherein the medical records include free text notes.

19. The computer-implemented method of claim 9, wherein the first trained machine learning model comprises a machine learning model that has been trained based on a plurality of natural images.

20. The computer-implemented method of claim 9, wherein generating the second trained machine learning model comprises using a supervised contrastive loss function to further train the first trained machine learning model.

21. The computer-implemented method of claim 20, wherein using the supervised contrastive loss function to further train the first trained machine learning model comprises using the supervised contrastive loss function with a temperature parameter greater than 0.5.

22. The computer-implemented method of claim 9, further comprising:
receiving an updated specific medical training data set, wherein the updated specific medical training data set includes an additional plurality of medical diagnostic images that are associated with the specific condition or diagnosis and a plurality of diagnostic labels associated therewith; and
using the updated specific medical training data set and the third trained machine learning model, generating an updated trained machine learning model by updating the third trained machine learning model, wherein the updated trained machine learning model is configured to receive an image as an input and to output, based on the input image, an output that is representative of a property or presence of the specific condition or diagnosis.

23. A computer-implemented method comprising:
receiving a first trained machine learning model, wherein the first trained machine learning model is configured to receive an image as an input and to output, based on the input image, an output vector that represents an embedding of the input image into a first multi-dimensional embedding space;
generating a second trained machine learning model by using a generic medical training data set to further train the first trained machine learning model, wherein the generic medical training data set includes a plurality of medical diagnostic images and a plurality of diagnostic labels associated therewith, wherein the second trained machine learning model is configured to receive an image as an input and to output, based on the input image, an output vector that represents an embedding of the input image into a second multi-dimensional embedding space;
receiving, by a first computing system from a second computing system, a target medical diagnostic image;
applying, by the first computing system, the target medical diagnostic image to the second trained machine learning model to generate a target output vector that represents an embedding of the target medical diagnostic image into the second multi-dimensional embedding space; and
transmitting, by the first computing system to the second computing system, an indication of the target output vector.

24. (canceled)

25. The computer-implemented method of claim 23, wherein the diagnostic labels of the plurality of diagnostic labels indicate whether their associated medical diagnostic images are normal or abnormal

26. The computer-implemented method of claim 25, further comprising:
generating the plurality of diagnostic labels based on medical records associated with the plurality of medical diagnostic images, wherein the medical records include free text notes

27. The computer-implemented method of claim 23, wherein the first trained machine learning model comprises a machine learning model that has been trained based on a plurality of natural images.

28. The computer-implemented method of claim 23, wherein generating the second trained machine learning model comprises using a supervised contrastive loss function to further train the first trained machine learning model.

29. The computer-implemented method of claim 28, wherein using the supervised contrastive loss function to further train the first trained machine learning model comprises using the supervised contrastive loss function with a temperature parameter greater than 0.5

30-30. (canceled)
